(19) United States
(12) Patent Application Publication
(10) Pub. No.: US 2009/0299285 A1
(43) Pub. Date: Dec. 3, 2009

(54) EXTENDABLE AUTO RETRACTABLE MEDICAL SYRINGE

(75) Inventors: Graeme Francis Walton, New South Wales (AU); Allan Walsh, New South Wales (AU)

Correspondence Address: BROOKS KUSHMAN P.C., 1000 TOWN CENTER, TWENTY-SECOND FLOOR, SOUTHFIELD, MI, 48075, US

(73) Assignee: GLOBAL MEDISAFE HOLDINGS PTY LIMITED, The Hill, NSW (AU)

(21) Appl. No.: 11/721,489

(22) PCT Filed: Apr. 8, 2005
(86) PCT No.: PCT/AU2005/000510

§ 371 Date: Jun. 12, 2007

(30) Foreign Application Priority Data
Dec. 14, 2004 (AU) 2004907100

(51) Int. Cl. A61M 5/50

(52) U.S. Cl. 604/110

(57) ABSTRACT

An auto retractable safety syringe (11) is disclosed. The syringe has barrel (12) which has a hollow plunger (13) located therein. The plunger has piston means (39) associated therewith so as to be sealingly slideable within the barrel. The syringe further has a needle cartridge assembly (14) located in the barrel, the needle cartridge assembly comprising a needle (20), needle hub (21), and a housing (15, 16) therefor. The needle cartridge assembly is initially releasably engaged on the end of the plunger in a retracted state within the barrel prior to use, and is movable to and engageable with the end (25) of the barrel where the needle extends therefrom in an in-use position for performing an injection. During this time the plunger is withdrawn from engagement with the cartridge, so as to function in known manner as a plunger for taking fluid into the barrel and causing it to be injected into a patient. Subsequent to completion of the injection, the plunger is depressed further to cause the cartridge to auto retract the needle such that it is caused to retract into the hollow plunger.

### TECHNICAL FIELD

This invention relates to a safety syringe having an auto retractable needle for use in the medical or dental profession or in personal drug administration so that the physician, surgeon or other needle operator might be protected from injury by the needle after its use.

### BACKGROUND

The danger of injury and possible infection from the HIV or hepatitis B virus to medical practitioners using needles in the normal course of their business is well documented.

Thus, there is a need to protect medical staff from needle stick injuries. In a time when it is known that there are many blood born diseases, it is becoming more critical to protect the medical profession against such diseases.

Patients also deserve the same protection against such diseases. If a medical practitioner receives a needle stick injury prior to giving an injection, any blood disease that medical practitioner has can be passed on to the patient.

Furthermore, persons who are in the habit of administering drugs to themselves run a severe risk of contacting either of the specified viruses, or indeed contacting other viruses if a needle, once used, is reused in an unsterilised form.

There have been many proposals aimed at reducing the number of so-called needle-stick injuries and various attempts have been made to provide a safe system for disposal of such needles once used, but such prior proposals have had deficiencies.

While it is known there are retractable syringes on the market, the syringe according to the present invention discussed herein, is not only auto-retractable, but is different in that the needle is retracted prior to use and is extended only at the time of being used.

This invention means there is no need for any medical practitioner to come in contact with the needle point, either before, during or after injection.

Because this syringe comes with the needle housed within the barrel, the needle remains sterile at all times.

### OBJECT OF THE INVENTION

It is therefore an object of the present invention to provide an auto retractable syringe, in which the needle not only retracts after use, but is in a retracted position prior to use. In this way, the syringe employs such a needle in a sterile manner prior to use, and provides for permanent storage of that surgical needle, once used, in a substantially safe manner.

It has been found that this object can be accomplished in a syringe design which utilises a cartridge to hold the needle prior to use, during injection, and which allows for the auto retraction of the needle to take place at the completion of the injection cycle.

Needle stick injuries are costly in both treatment and lost working time. This invention will also greatly reduce the spread of dangerous diseases saving lives and millions of dollars in medical treatments.

At the very least the invention provides an alternate means for providing an auto retractable syringe to protect against accidental injury arising from unwanted contact with the exposed needle prior to use and once the syringe has been used.

Although the following description generally refers to a syringe of conventional size, no such limitation is intended thereby, and reference to a syringe is meant to encompass any other needle/syringe combination or needle alone including slimline syringes, where, by suitable adaptation, the invention may also be usefully applied.

### DISCLOSURE OF THE INVENTION

The invention in one broad form provides an auto retractable safety syringe having a syringe barrel which has a hollow plunger located therein, the plunger having piston means associated therewith so as to be sealingly slideable within the barrel, the syringe further having a needle cartridge assembly located in the barrel, the needle cartridge assembly comprising a needle, needle hub, and a housing therefor, the needle cartridge assembly being initially releasably engaged on the end of the plunger in a retracted state within the barrel prior to use, and movable to and engageable with the end of the barrel where the needle extends therefrom in an in-use position for performing an injection, and during which time the plunger is withdrawn from engagement with the cartridge, so as to function in known manner as a plunger for taking fluid into the barrel and causing it to be injected into a patient, and wherein subsequent to completion of the injection the plunger is depressed further to cause the cartridge to auto retract the needle such that it is caused to retract into the hollow plunger.

It will be appreciated that in order to function in the normal way, the hollow plunger will be initially sealed for example by a disc in the form of a seal across the end of the plunger, which seal will be broken or ruptured during auto retraction. Preferably the seal is made of a flexible rubber or rubber like material.

Preferably the means to auto-retract the needle is a spring located about the needle which remains in a compressed state prior to and during use, and which upon completion of the injection is caused to expand by virtue of the action of the plunger on the cartridge causing auto retraction to occur.

The plunger is thus required to first locate the cartridge in the in-use position during which the plunger is withdrawn and operated in the normal way by causing fluid to be drawn into the syringe and then injected into a patient and to then subsequently activate the auto retract facility by forcing the cartridge to collapse under further pressure. In order to facilitate this required two stage action of the plunger, it is preferred that the synthetic piston slides on the plunger by way of grooves within the bore of the piston that interact with the ridge located about the plunger, thus allowing the first and second stages to be achieved.

To this end, one preferred embodiment comprises a collapsible housing for the cartridge, with the spring located between the needle hub and the base of the housing, so that upon collapse of the housing effected by the plunger being further compressed to its second stage, the needle hub is released from engagement within the housing, thereby releasing the needle and allowing the spring to expand, further causing the needle hub and needle to be relocated in the hollow plunger. It will be understood that the spring pressure will need to be sufficient not only to move the needle and hub thus, but also to break or rupture the disc sealing the hollow plunger.

Preferably the housing has portions which are slideable in relation to each other. Thus for example the first part is a needle support boss which engages the hub of the needle prior to and during injection. Preferably engagement is by means of a simple taper, where a taper is provided on the needle hub which engages with a corresponding taper in the needle support boss.

The second portion is preferably a lower support bush or sleeve, located in or on the support boss and slideable in relation thereto.

Initially, the two portions will be extended under the pressure of the spring. However, when the plunger is further compressed during the auto retraction stage, the portions will be caused to slide together, it be compressed, causing the hub to be freed from engagement with the support boss and allowing the spring to act as it then able to expand.

Preferably the means to retain the cartridge in the end of the barrel during injection, is provided by means of a groove about the internal periphery of the barrel which mates with a corresponding ridge or flange located about the perimeter of the cartridge. Preferably means are provided to retain the collapsed cartridge within the barrel in its compressed state, thus rendering the syringe inoperative. Such means include a second groove about the internal periphery of the barrel for mating with the aforementioned ridge or flange.

Thus the needle hub and needle is initially connected to the plunger as part of a cartridge assembly. The needle is initially protected within the barrel prior to use and is only caused to extend from out of the end of the barrel at the time the syringe is about to be used for an injection. In the preferred form of the invention, the cartridge is made up of a compression spring, needle hub and needle, needle support boss with taper to hold needle hub, and a support sleeve.

In a particularly preferred form of the invention, attention has been given to the rate at which the auto retraction is allowed to occur. Without adequate control, there can be undesirable splash back from fluid retained in the needle. Whilst at first it might be thought useful to simply reduce the strength of the spring, in practice this is not feasible since the spring has to be sufficient to move the needle and hub back into plunger along with the plunger seal.

In order to slow the retraction, at least initially, to a rate at which splatter is less likely to occur three methods have been found useful all of which rely on the principle of controlling the airflow in the plunger as the needle etc is retracted. Thus when the needle hub is dislodged at the time of compression, the spring pressure pushes the needle hub up pushing the already dislodged inner seal inside the plunger.

One method of controlling the airflow is to provide a taper within the inside of the hollow plunger, so that the needle hub is caused to start its journey more slowly by virtue of the initial greater resistance. The second method is to provide a bleed hole at the upper end of the plunger, whereby the air can be allowed to escape, thus reducing the likelihood of pressure build up in the plunger which would otherwise only be released through the needle itself, thus causing splatter. Finally the other method is to provide one or more grooves in the periphery of the plunger seal so that air is allowed to be released thereby reducing the pressure in the plunger and hence the possibility of splashback.

Thus the inner seal sealing the plunger in a preferred embodiment has air grooves cut through to allow a slow return of the needle inside the plunger eliminating blood splash at the time of auto needle retraction after injection has taken place.

A more sophisticated solution utilises a tapered hole in the plunger. When inner seal is retracted by syringe pressure after injection, the tapered bore allows ever increasing air to escape around the inner seal. This allows the needle retraction to start slowly but will cause the needle to increase in velocity.

### BRIEF DESCRIPTION OF THE DRAWINGS

The invention may be better understood from the following non-limiting description of preferred embodiments, in which:

FIG. 1 is a cross sectional exploded side view of a needle and syringe combination having a needle cartridge for auto retraction of the needle after use according to one embodiment of the invention;

FIG. 2 is a cross sectional exploded side view of a second needle cartridge according to an alternate embodiment of the invention;

FIG. 3 shows the detail of the end of an alternate plunger according to a further embodiment of the invention;

FIG. 4 is a cross sectional view of an assembled needle and syringe of FIG. 1 before use;

FIG. 5 is a cross sectional view of an assembled needle and syringe of FIG. 1 ready for use;

FIG. 6 is a cross sectional view of an assembled needle and syringe of FIG. 1 after an injection has been completed; and

FIG. 7 is a cross sectional view of an assembled needle and syringe of FIG. 1 after auto retraction has taken place;

### DETAILED DESCRIPTION OF THE DRAWINGS

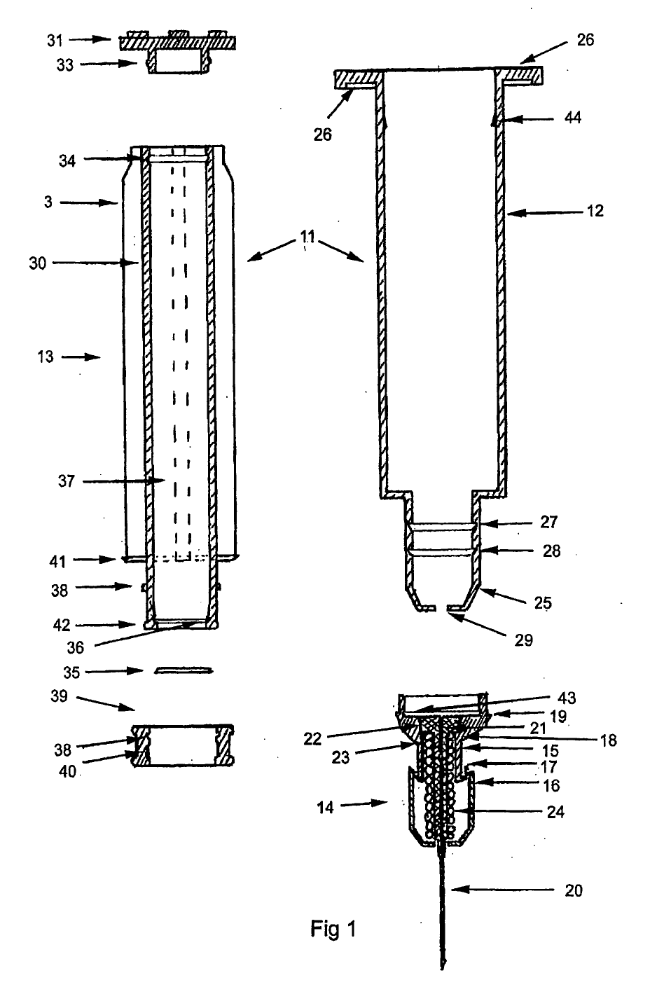

Referring generally to the Figures and in particular to FIG. 1, there is shown a syringe and needle combination generally referenced 11, which comprises a syringe barrel 12, a hollow plunger 13 and a needle cartridge 14.

Needle cartridge 14 comprises a hollow needle support boss 15 and lower support bush 16 slideably located thereon. The cartridge is able to collapse to the extent that the lower support bush 16 is able to move upwards on the boss 15, ie as far as it is able by virtue of the shape of the boss 15. The needle support boss has shoulder 17 against which end 18 of the lower bush 16 comes into contact when the cartridge 14 is collapsed during auto retraction as described below.

The needle support boss 15 has a ridge 19 located about its periphery. The needle support boss 15 accommodates a needle 20 having a tapered needle hub 21 at its upper end. There is a corresponding tapered portion 22 located in the needle support boss 15 to initially hold the needle hub 21. This taper 22 has a limited depth to prevent the needle hub 21 from locking too tightly in the taper 22. This is achieved by a shoulder 23 located at the base of the taper 22 in the needle support boss 15.

Located about needle 20 and contained within the pre-assembled cartridge 14, there is a compression spring 24. The inside of boss 15 is hollow to initially accommodate the needle 20 and the compression spring 24 during injection and then allow free passage thereof after injection and at the time of auto retraction as described below.

Syringe barrel 12 is a hollow cylinder with a reduced end 25 to accommodate the needle cartridge 14. Around the circumference of the barrel 12 there may be provided graduations (not illustrated) to measure fluid level. At the top of the barrel 12 are two wings 26 extending perpendicular to the cylindrical shape for use as finger grips.

Within the reduced end portion 25 of the barrel 12 there are two grooves 27 and 28. Groove 27 mates with ridge 19 located on the needle boss 15 of needle cartridge 14, in order to hold the needle 20 extended during injection as explained below.

The second groove 28 is the needle cartridge locking groove. When auto retraction has been activated as described below, the needle cartridge 14 is collapsed and boss ridge 19 moves down to cartridge locking groove 28, thereby locking the needle cartridge 14 and rendering it unusable.

A small hole 29 is provided in the end of the barrel 12 to allow the end of the needle 20 to protrude at the time of using the syringe 11 to give an injection.

The barrel 12 also accommodates hollow plunger 13 which has four flutes 30 equally spaced around the circumference to act as guides when in the barrel 12. The flutes also add strength to the plunger 13.

A plunger cap 31 has raised finger grips 32 on the upper edge to give assistance to the user to control the handling of the syringe 11. A raised ridge 33 on the cap 31 clips into the plunger 13 at corresponding groove 34 located therein. This adds ease of assembly and holds the cap 31 into the plunger 13 firmly. The plunger cap 31 is also hollowed out to reduce weight.

At its lower end, the plunger 13 is provided with a rupture disc 35 which is fitted in rupture disc groove 36 and initially seals the hollow interior 37 of the plunger 13. After the syringe 11 has been used and auto retraction has taken place as described in further detail below, the hollow centre 37 of plunger 13 accommodates the rupture disc 35, the needle 20 and needle hub 21, along with the compression spring 24.

Rupture disc 35 is a flat disc with a ‘vee’ section on the circumference to hold it in the groove 36. The rupture disc 35 is smaller in diameter than the bore of the plunger 13 to allow free passage after injection is complete, at the time of auto retraction. The end of the plunger 13 in the region of groove 36 is in fact reduced in diameter to thus hold the rupture disc 35.

Around the lower periphery of the plunger 13, there is located piston ridge 38 which holds the piston 39 in place prior to injection and until auto retraction takes place, at which time the piston 39 is forced up the plunger 13 when the piston 39 bottoms out in the barrel 12. This action of the plunger 13 forces the piston 39 to slide into the elongated groove 40 in the centre of the piston 39. The piston 39 is then located against flange 41.

The piston 39 seals between the plunger 13 and the bore of the syringe barrel 12. When injection is complete, the piston 39 bottoms in the barrel and as pressure is exerted, the plunger 13 slides through the piston 39 into the recess 40 of the piston 39. This allows auto retraction to take place as explained below.

On the outside of the lower portion of the plunger 13 is the cartridge matching ridge 42. This ridge 42 mates with corresponding recess 43 located in the needle support boss 15 thereby allowing the cartridge 14 to be held on the plunger 13 prior to use, at which time the cartridge 14 and hence needle 20 are held inside the barrel 12.

After the plunger 13 is initially pushed down, ie ready for use, so that the cartridge 14 is located in the barrel end 25 and the cartridge 14 is thus in the extended position, ie the needle 20 extends from the barrel 12 through hole 29, the plunger 13 is then retracted allowing the separation of the cartridge 14 from the plunger 13 and leaving the cartridge 14 held in the end 25 of the barrel 12, by virtue of engagement of ridge 19 on the needle support boss 15 with first groove 27.

After completing the injection, the further downward push of the plunger 13 at the time of auto retract causes the needle support boss 15 to be forced down within the lower support bush 16 reducing the overall height of the cartridge 14. This breaks the connection between the taper 22 in the boss 15 and the tapered needle hub 21, causing the needle 20 and hub 21 to be released and under the influence of the compression spring 24, being now free, are pushed along with the freed rupture disc 35 into the hollow inside 37 of the plunger 13. At this time auto retraction has taken place.

When the auto retract has thus been activated, the needle cartridge 14 having been collapsed, the boss ridge 19 comes in contact with cartridge locking groove 29. This locks the needle cartridge 14 in the barrel 12 in its collapsed state, thereby rendering it unusable.

There is also provided a tapered ridge 44 inside the barrel 12. The plunger 13 is forced over the ridge 44 but when the plunger 12 is retracted, the tapered flange 41 comes in contact with ridge 44 preventing the plunger 13 being removed.

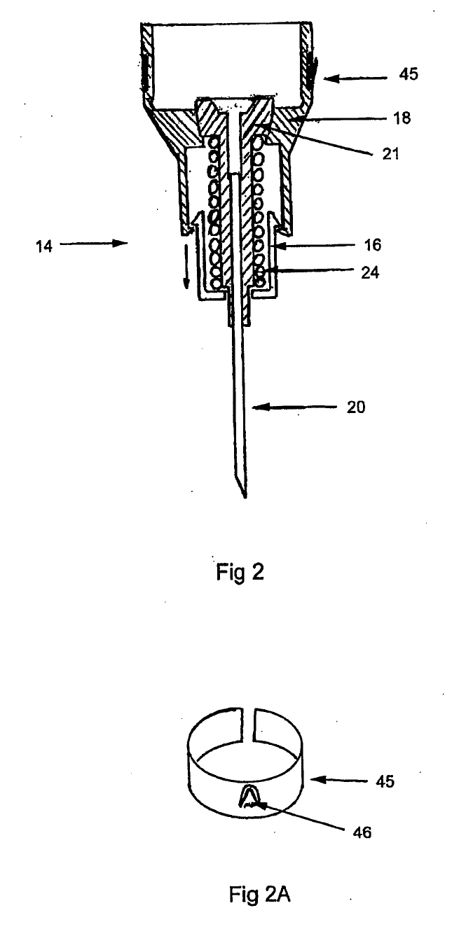

Referring to FIG. 2, there is shown an alternate embodiment of the needle cartridge 14, where similar features are identified with the same reference numerals as those exemplified in FIG. 1. The primary differences here are that the needle support boss 15 and lower support bush 16 are reversed in so far as the latter slides within rather than over the former. And that the boss ridge 19 is relaced with a stainless steel clip 45, the detail of which is shown in FIG. 2A. The clip 45 is a simple cir-clip having a protrusion 46 in the form of a cut out which provides for simpler but effective holding of the cartridge 14 in the respective in-use (ie injection) and later storage (ie after auto retraction) positions.

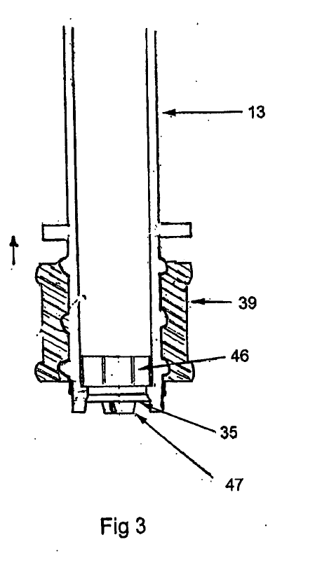

Referring to FIG. 3, there is shown in detail a portion of the end of a plunger, in which again like components to those already described are reference with the same numerals. In this case, the inner seal 47 relaces the simpler rupture disc 35. Here the seal 45 has air grooves 46 cut in the periphery to allow the egress of air during auto retraction to control the effect thereof as described above. This embodiment also shows the needle hub spigot which mated with the top of the needle hub 21 as shown in the earlier drawings.

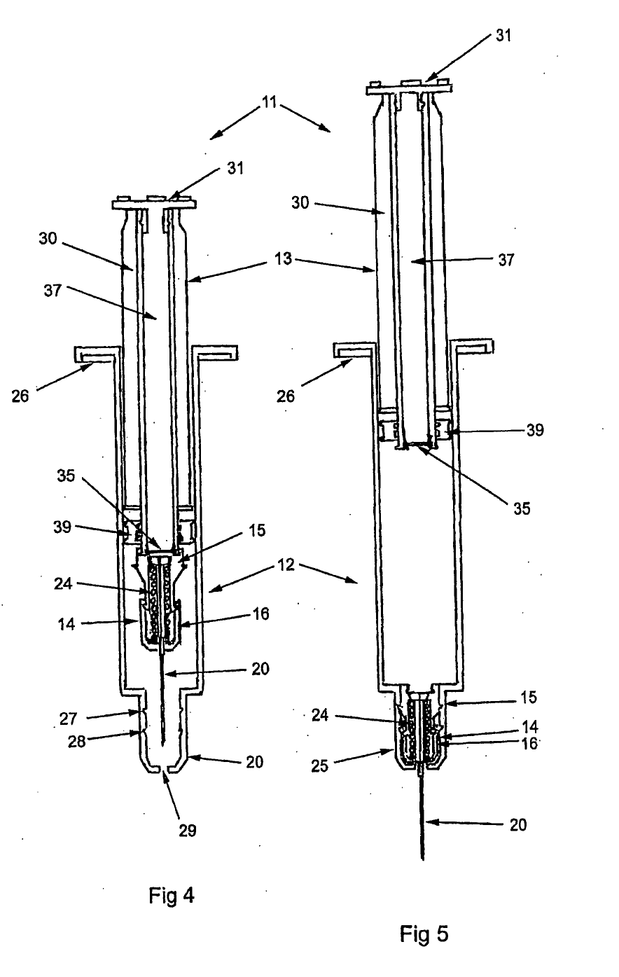

Referring generally to FIGS. 4 through 7, there are shown the various stages in the use of the syringe 11. The syringe 11 comes sealed in a sterile package (not shown). Outlined on the packaging is the size of the syringe along with the needle size.

In FIG. 4, the needle cartridge 14 is attached to the plunger 13 inside the syringe barrel 12.

The plunger 13 is then pushed down to install needle cartridge 14 into the end of the syringe barrel 12. This has taken place when a first click is felt. The needle 20 will now be extended through the hole 29 in end of the barrel 12 and is now ready for use. The plunger 13 is then retracted to break contact between the plunger 13 and the needle cartridge 14. With the needle 20 inserted in the vial of medical solution, the plunger is then drawn further back to draw up the required amount of fluid, as shown in FIG. 5.

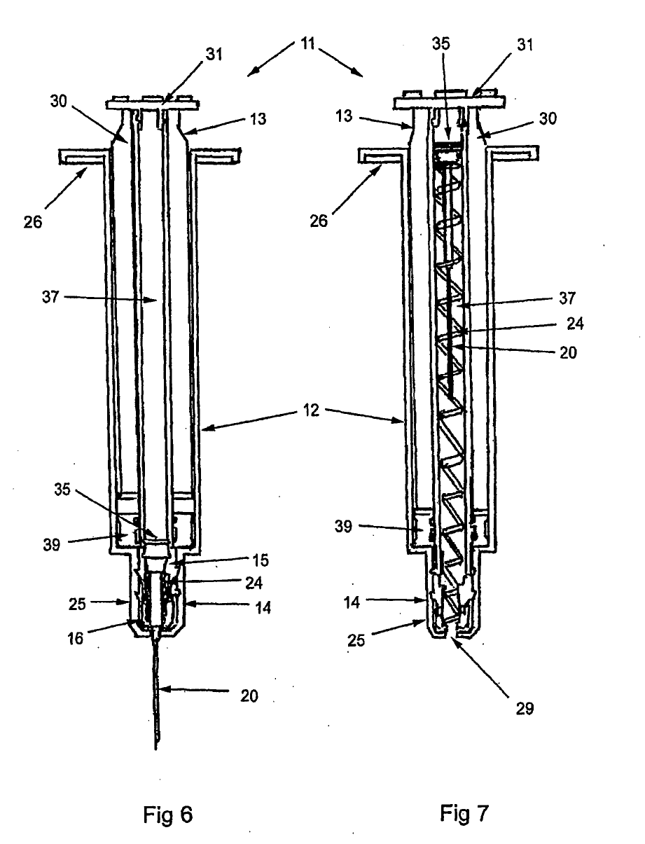

The injection is then given by pushing the plunger 13 down. By pushing further on the plunger 13, a further click will be felt (see FIG. 6).

When the cartridge 14 reduces in height (as described above), the taper between the needle support boss 15 and the tapered needle hub 21 is released and the spring 24 pushes the needle hub 21 into the plunger 13, which carries the needle 20 into the plunger 13 as well, pushing the freed rupture disc 35 in front of the needle hub 21 (see FIG. 7).

The retracted syringe may then be disposed of safely in the sharps container, the needle safely contained within the plunger and hence within the barrel 12, the syringe 11 also being made inoperative by virtue of the retention of the collapsed cartridge within the end 25 of the barrel 12.

It will be appreciated by those skilled in the art that many modifications and variations may be made to the embodiments described herein without departing from the spirit or scope of the invention.

Throughout the specification the word “comprise” and its derivatives are intended to have an inclusive rather than exclusive meaning unless the context requires otherwise.

1. An auto retractable safety syringe having a syringe barrel which has a hollow plunger located therein, the plunger having piston means associated therewith so as to be sealingly slideable within the barrel, the syringe further having a needle cartridge assembly located in the barrel, the needle cartridge assembly comprising a cartridge housing a needle having a needle hub, the needle cartridge assembly further having auto retractable means whereby the needle may be ejected from the needle cartridge assembly and fully retracted into the hollow plunger upon activation of the auto retractable means, the needle cartridge assembly being initially releasably engaged on the end of the plunger in a retracted state within the barrel prior to use, and movable to and engageable with the end of the barrel where the needle extends therefrom in an in-use position for performing an injection, and during which time the plunger is withdrawn from engagement with the needle cartridge assembly, so as to function in known manner as a plunger for taking fluid into the barrel and causing it to be injected into a patient, and wherein subsequent to completion of the injection the plunger is depressed further to cause the needle cartridge assembly to eject the needle from the needle cartridge assembly by activating the auto retractable means such that the needle is caused to retract into the hollow plunger.

2. An auto retractable safety syringe according to claim 1, in which the hollow plunger is initially sealed by a disc in the form of a seal across the end of the plunger, which seal is broken or ruptured during auto retraction.

3. An auto retractable safety syringe according to claim 2, in which the seal is made of a flexible rubber or rubber like material.

4. An auto retractable safety syringe according to claim 1, in which the auto retractable means to eject the needle is a spring located about the needle which remains in a compressed state prior to and during use, and which upon completion of the injection is caused to expand by virtue of the action of the plunger on the needle cartridge assembly causing auto retraction to occur.

5. An auto retractable safety syringe according to claim 4, wherein the cartridge of the needle cartridge assembly is a two piece cartridge and wherein the plunger is required in a first stage to locate the needle cartridge assembly in the in-use position during which the plunger is withdrawn and operated in the normal way by causing fluid to be drawn into the syringe and then injected into a patient and to then in a second stage subsequently activate the auto retract facility by forcing the two piece cartridge to collapse under further pressure.

6. An auto retractable safety syringe according to claim 5, in which a synthetic piston slides on the plunger by way of having grooves within the bore of the piston that interact with a ridge located about the plunger, thus allowing the first and second stages to be achieved.

7. An auto retractable safety syringe according to claim 5, in which the cartridge is a collapsible housing, with the spring located between the needle hub and the base of the housing, so that upon collapse of the housing effected by the plunger being further compressed in the second stage, the needle hub is released from engagement within the housing, thereby releasing the needle and allowing the spring to expand, further causing the needle hub and needle to be relocated in the hollow plunger.

8. An auto retractable safety syringe according to claim 7, in which the housing has portions which are slideable in relation to each other.

9. An auto retractable safety syringe according to claim 8, in which a first part of the housing is a needle support boss which engages the hub of the needle prior to and during injection.

10. An auto retractable safety syringe according to claim 9, in which engagement of the needle hub with the boss is by means of a simple taper, where a taper is provided on the needle hub which engages with a corresponding taper in the needle support boss.

11. An auto retractable safety syringe according to claim 9 wherein the second portion of the needle cartridge housing is a lower support bush or sleeve, located in or on the support boss and slideable in relation thereto.

12. An auto retractable safety syringe according to claim 8, in which initially, the two portions of the housing will be extended under the pressure of the spring, and when the plunger is further compressed during the auto retraction stage, the portions will be caused to slide together, causing the needle hub to be freed from engagement with the support boss and allowing the spring to act thereon by virtue of it being able to expand.

13. An auto retractable safety syringe according to claim 1, in which the means to retain the needle cartridge assembly in the end of the barrel during injection is provided by means of a groove about the internal periphery of the barrel which mates with a corresponding ridge or flange located about the perimeter of the cartridge of the needle cartridge assembly.

14. An auto retractable safety syringe according to claim 13, in which means are provided to retain the cartridge within the barrel after auto retraction, thereby rendering the syringe inoperative, the means being provided by a further groove about the internal periphery of the barrel for mating with the aforementioned ridge or flange.

15. An auto retractable safety syringe according to claim 1, in which the rate of travel of the needle and hub during auto retraction is controlled by controlling the flow of air in the hollow plunger, in order to reduce so called splash back.

16. An auto retractable safety syringe according to claim 15, wherein the method of controlling the airflow is to provide a taper within the inside of the hollow plunger, so that the needle hub is caused to start its journey more slowly by virtue of the initial greater resistance.

17. An auto retractable safety syringe according to claim 15, wherein the method of controlling the airflow is to provide a bleed hole at the upper end of the plunger, whereby the air can be allowed to escape, thus reducing the likelihood of pressure build up in the plunger which would otherwise only be released through the needle itself, thus causing splatter.

18. An auto retractable safety syringe according to claim 15, wherein the method of controlling the airflow is to provide one or more grooves in the periphery of the plunger seal so that air is allowed to be released thereby reducing the pressure in the plunger and hence the possibility of splashback.

19. (canceled)

20. An auto retractable safety syringe according to claim 2, in which the auto retractable means to eject the needle is a spring located about the needle which remains in a compressed state prior to and during use, and which upon completion of the injection is caused to expand by virtue of the action of the plunger on the needle cartridge assembly causing auto retraction to occur.

21. An auto retractable safety syringe according to claim 3, in which the auto retractable means to eject the needle is a spring located about the needle which remains in a compressed state prior to and during use, and which upon completion of the injection is caused to expand by virtue of the action of the plunger on the needle cartridge assembly causing auto retraction to occur.
